(19) United States
(12) Patent Application Publication
(10) Pub. No.: US 2008/0176205 A1
(43) Pub. Date: Jul. 24, 2008

(54) Process and Formulation to Improve Viability of Stored Cells and Tissue

(75) Inventor: Jane Shelby, Bozeman, MT (US)

Correspondence Address: NEEDLE & ROSENBERG, P.C., SUITE 1000, 999 PEACHTREE STREET, ATLANTA, GA, 30309-3915, US

(73) Assignee: UNIVERSITY OF UTAH RESEARCH FOUNDATION, Salt Lake City, UT (US)

(21) Appl. No.: 10/581,386

(22) PCT Filed: Dec. 6, 2004
(86) PCT No.: PCT/US2004/040824

§ 371 Date: Mar. 6, 2008

Related U.S. Application Data
(60) Provisional application No. 60/526,909, filed on Dec. 4, 2003.

(51) Int. Cl. A01N 1/02; C12N 5/00; C12N 5/02

(52) U.S. Cl. 435/1.1; 435/325; 435/374; 435/1.3; 435/404

(57) ABSTRACT

Methods and compositions for the preservation of cells using glucosaminoglycans and derivatives thereof.

1. This application claims priority to U.S. provisional application number 60/526,909, filed on Dec. 4, 2003. The aforementioned application is herein incorporated by this reference in its entirety.

### I. BACKGROUND

Transplantation of human organs and tissues saves many lives and restores essential functions in circumstances when no medical alternative of comparable effectiveness exists. The transplantation of solid organs, such as kidney, liver, heart or lung, is increasingly a regular component of health care.

The tissues, organs, and cells used in transplantation are carefully removed from the donor, appropriately stored, and prepared for transplantation. Methods currently employed for the preservation of cellular biological materials include immersion in saline-based media; storage at temperatures slightly above freezing; storage at temperatures of about −80° C.; and storage in liquid nitrogen at temperatures of about −196° C. The goal of all these techniques is to store living biological materials for an extended period of time with minimal loss of normal biological structure and function. Traditional cryopreservation of skin is associated with damage to epithelial cells and lower viability. Availability of higher quality viable tissue would result in faster healing and significant savings of treatment costs.

Many media formulations for specific cells and tissue culture have been discussed in the art. Examples include Gardner D K, Rodriegez-Martinez H and Lane M. 1999 Hum Reprod 14 (10):2575-84; Stojkovic M, Thompson J G and Tervit, H R. 1999 Theriogenology 51: 254; Tammi R, Saamanen A-M, Maibach H I and Tammi M. 1991 J Invest Dermatol 97:126-130; Poggi M M, Klein M B, Chapo G A, Cuono C B. 1999 J Burn Care Rehabil 20 (3):201-6.) Preservation solutions are disclosed in U.S. Pat. Nos. 6,548,297 (Kari-Haruch); 5,071,741 (Brockbank); 5,131,850 (Brockbank); and published U.S. application U.S.2001/0009908 (Ponzin) as well as Hovatta et al., 12 Hum. Reprod. 1032 (1997), each of which is disclosed herein at least for material related to preservation solutions.

There thus remains a need in the art for improved methods for the storage and preservation of living biological materials that more effectively maintain the integrity, viability and function of all types of cells during the cryopreservation process.

### II. SUMMARY OF EMBODIMENTS

Described herein are methods and compositions for the preservation of cells, tissues, and organs using glucosaminoglycans and derivatives thereof.

### III. BRIEF DESCRIPTION OF THE DRAWINGS

The accompanying drawings, which are incorporated in and constitute a part of this specification, illustrate several aspects described below.

FIG. 1 shows the mean dissolved O₂ concentrations in HA enriched media. The media preserved viability of the human skin, assessed by O₂ consumption at 7 and 10 days post harvest (p<0.001 vs media controls).

FIG. 2 shows sterile cadaveric human skin cultured in various media formulations for varied times post harvest (stored and cryopreserved), which is cut to a size equal to the wound on the athymic nude mouse. After the wound has been made, the cadaveric skin is immediately placed on the wound and sutured into place. Laser Doppler ultrasound is used to determine cutaneous blood flow in healing skin, assessing potential faster engraftment of skin cultured in HA enriched media. Biopsies of the human skin graft are taken at 1, 2, and 4 weeks post transplant, and assessed histologically for structural integrity.

FIG. 3 shows three graphs at Day 9, Day 15, Day 18 (FIG. 3A-C). Oxygen consumption of human skin explants cultured in RPMI only (Control), RPMI+HA 200 k Da 1 mg/ml (HA) or RPMI+CS 1 mg/ml (CS) is shown. At Days 9, 15 and 18 of in vitro culture at 4° C., skin immersed in RPMI supplemented with HA or CS had a greater consumption of O₂, indicating greater metabolic activity and viability.

FIG. 4 shows two graphs (4A and 4B) at Day 21 and Day 25. Oxygen consumption of human skin explants cultured in RPMI only (Control), RPMI+HA 200 kDa 1 mg/ml (HA) or RPMI+CS 1 mg/ml (CS) is shown. At Days 21 and 25 of in vitro culture at 4° C., skin immersed in RPMI supplemented with HA or CS had a greater greater consumption of O₂, indicating greater metabolic activity and viability.

FIG. 5 shows one graph at Day 18. Oxygen consumption of human skin explants cultured in RPMI only (Control), RPMI+HA 200 kDa 1 mg/ml (HA) is shown. At Day 18 of in vitro culture at 4° C., skin immersed in RPMI supplemented with HA had a greater greater consumption of O₂, indicating greater metabolic activity and viability.

FIG. 6 shows oxygen consumption of human skin explants cultured in RPMI only (Control), RPMI+HA 200 kDa 1 mg/ml (HA). At Day 9 of in vitro culture at 4° C., skin immersed in RPMI supplemented with HA had a greater consumption of O₂, indicating greater metabolic activity and viability, with larger size HA and greater concentration suggesting a more robust effect. Similar viability promoting effects have been observed with 1700 kDa HA and other sizes.

FIG. 7 shows HA is an effective cryoprotectant. A trial was conducted comparing the viability of preadipocyte cells after cryopreservation. The cells were stored in HA, DMEM, FBS, or FBS/HA. The cells were compared at pre-freeze, then again post-thaw. The results show that HA or FBS/HA was superior to DMEM alone.

FIG. 8 shows a cell trial comparing graded concentrations of HA. DMEM/FBS was compared to 2%, 1%, 0.5%, 0.1%, and 0% HA. The best results are obtained using 1-2% HA.

FIG. 9 shows post thaw cell viability when various concentrations of HA are used. DMEM/FBS was compared to 2%, 1%, 0.5%, 0.1%, and 0% HA

FIG. 10 shows the viability of cryopreserved cells with graded HA concentrations. DMEM/FBS is compared with 4%, 2%, 1%, and 0% HA. This assay shows that a variety of concentrations of HA can be used to maintain the viability of cryopreserved cells.

### IV. DETAILED DESCRIPTION

### A. Definitions

Before the present composites, compositions, and/or methods are disclosed and described, it is to be understood that the aspects described below are not limited to specific compounds, synthetic methods, or uses as such may, of course, vary. It is also to be understood that the technology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a, ” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

“Cellular matter” or “cell” refers to a living structure, composed of a mass of protoplasm, enclosed in a membrane and containing a nucleus. It may or may not be part of a larger structure, such as a tissue or an organ. Where the term “cell” is used, it is understood that “tissues” or “organs” can be substituted.

“Osmotic effects” refers to the alteration in the osmotic strength of the suspending media caused by conversion of water to ice or ice to water. This conversion results in substantial flow of water across membranes of unfrozen cells, causing volume changes during freezing and thawing.

“Viability” refers to the ability of frozen and thawed cells to perform their normal functions. Viability is usually expressed as the ability of the cells to reproduce, metabolize, exclude vital dyes or carry out some other metabolic function. The viability of the frozen and thawed samples can be compared to the ability of unfrozen cells obtained at the same time to carry out the same function.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Compositions and methods

Methods for the preservation of biological materials are employed in many clinical and veterinary applications wherein living material, including organs, tissues and cells, are harvested and stored in vitro for some period of time before use. Examples of such applications include organ storage and transplants, autologous and allogeneic bone marrow transplants, whole blood transplants, platelet transplants, cord blood and other stem cell transplants, embryo transfer applications such as those used in in vitro fertilization, embryonic stem cell storage, skin grafting, and storage of tissue biopsies for diagnostic purposes.

Preservation techniques are also important in the storage of cell lines for experimental use in hospital, industrial, university and other research laboratories. For instance, preserved cells are often tested to aid in the development of medical treatments or to provide information on physical or chemical properties of the cells. Further, a collection of readily available viable cells allows scientists to conduct experiments at optimal times. To be useful, the preserved cells should retain the integrity and viability of the cells at the time of harvest. These uses include medical, veterinary, and research other research uses.

In conventional cryopreservation techniques, cells are harvested, suspended in a storage solution, then preserved by freezing. When the cells are to be used, they are thawed, for example, cells taken from human donor sources are brought back to the normal human body temperature (i.e., approximately 37° C.), and then placed in a cell culture medium. Cryopreservation protocols subject the cells to a multitude of stresses and insults throughout the process of cell harvesting, freezing, and thawing. These stresses and insults can cause irreparable damage to the cell.

Ischemia, a lack of blood flow, occurs as soon as the life of the cell's donor is terminated immediately thereafter, the cell experiences hypoxia, or oxygen deprivation, due to the lack of blood flow. Hypoxia causes anaerobic metabolism in normally aerobic cells. Anaerobic metabolism produces toxic byproducts, such as the build-up of lactic acid (acidosis). Some of the byproducts of anaerobic metabolism produce oxygen free-radicals that damage or destroy the cells when the cells are reoxygenated. Accordingly, prior to taking a tissue sample, the temperature of the donor source is reduced such that metabolic activity in the cells of the donor source is minimized. Reduction of temperature of the donor source reduces the energy state of the cells which aids in reducing the affects of ischemia and hypoxia. Typically, the temperature of the donor source is lowered to 4° C. Similarly, donor whole organs are placed in solutions at 4° C. prior to transplantation. Although some residual metabolic activity exists in the cells at 4° C., it is about the lowest temperature available which does not cause the formation of ice crystals on the cells.

Cadaveric allograft skin is often used for temporary coverage of full thickness burn wounds, as well as in the treatment of chronic ulcers. These grafts facilitate the early excision of bum injuries, providing a protective barrier function, reduce microbial colonization, and hasten the preparation of the graft bed for final coverage with autologous skin grafts. Allograft skin stored short term in media at 4° C. is clinically superior to cryopreserved skin. However, non-cryopreserved allograft skin that is cultured greater than 96 hours rapidly loses viability due to oxidative processes and is therefore not useful in all applications. Cadaveric skin is also used for research in a variety of studies including transdermal delivery of drugs.

Described herein are improved methods and compositions to increase viability and shelf life of cells, tissues, and organs. An optimized media formulation was developed using an extracellular matrix (ECM) glycosaminoglycan (GAG), as an alternative to serum, or along with serum, to provide superior viability and function results.

Thus, disclosed are compositions that comprise GAG and some type of tissue, organ, or cell.

### 1. Tissue Preservation Solutions

Disclosed are tissue preservation compositions that comprise a GAG, such as HA. The compositions can also comprise a media for preservation of tissues. The compositions can also comprise a number of other materials, such as cryopreservation agents, antibiotics, and the like. Also disclosed are compositions that do not contain serum.

The compositions are useful for maintaining the tissues for preservation, for example, prior to cryopresevation. The compositions are capable of extending the useful properties of the tissue, in solution, prior to going into cryopreservation and, for example, after the tissues come out of cryopreservation. Also disclosed are methods of using the disclosed compositions to prepare tissues prior to cryopreservation and storage before cryopreservation, as well as after the tissues come out of cryopreservation. The time of non-cryo storage can vary. The GAGs can be used at a variety of concentrations.

The preserving compositions containing a GAG can be found in the presence or in the absence of serum. If desired, serum formulations available in the art may be added to the medium, such as fetal bovine serum, for example at a concentration between about 10% and 40% (volume percent), or bovine neonatal serum. It is typically preferable that no serum be used. Serum free media allows for the reduction of disease transmission, and less variability, because various batches of sera can contain different levels of cytokines and hormones that have various effects on the cells and tissue. In some embodiments, serum is included in the freezing medium containing arabinogalactan together with additional permeating cryoprotective agent such as DMSO. The medium can be readily adjusted for a particular cell sample.

Disclosed are compositions for preserving cells comprising non-cultured, non-corneal, non-ovarian and non-musculoskeletal cells and a GAG, such as hyaluronan, in the absence of serum and in the absence of a non-cell penetrating cryoprotectant. Also disclosed are compositions for preserving cells comprising non-comeal, non-ovarian and non-musculoskeletal cells and a GAG, such as hyaluronan, in the absence of serum and in the absence of a non-cell penetrating cryoprotectant in which the cells are preserved at a temperature of above 0° C.

Disclosed are compositions for preserving cells comprising non-cultured epithelial cells and a GAG, such as hyaluronan, in the absence of serum and in the absence of a non cell-penetrating organic solute. Also disclosed are compositions for preserving cells comprising epithelial cells and a GAG, such as hyaluronan, in the absence of serum and in the absence of a non cell-penetrating organic solute in which the cells are preserved at a temperature of above 0° C.

Disclosed are methods of making a storage solution comprising adding non-cultured, non-corneal, non-ovarian and non-musculoskeletal cells to a GAG, such as hyaluronan, in the absence of serum and in the absence of a non-cell penetrating cryoprotectant.

Also disclosed are methods of making a storage solution comprising adding non-corneal, non-ovarian and non-musculoskeletal cells to a GAG, such as hyaluronan, in the absence of serum and in the absence of a non-cell penetrating cryoprotectant in which the cells are preserved at a temperature of above 0C.

Disclosed are methods of preserving cells comprising storing non-cultured, non-corneal, non-ovarian and non-musculoskeletal cells and a GAG, such as hyaluronan, in the absence of serum and in the absence of a non-cell penetrating cryoprotectant.

Also disclosed are methods of preserving cells comprising non-corneal, non-ovarian and non-musculoskeletal cells and a GAG, such as hyaluronan, in the absence of serum and in the absence of a non-cell penetrating cryoprotectant in which the cells are preserved at a temperature of above 0° C.

Disclosed are methods of treatment comprising storing non-cultured, non-corneal, non-ovarian and non-musculoskeletal cells and a GAG, such as hyaluronan, in the absence of serum and in the absence of a non-cell penetrating cryoprotectant.

Disclosed are methods of treatment comprising non-corneal, non-ovarian and non-musculoskeletal cells and a GAG, such as hyaluronan, in the absence of serum and in the absence of a non-cell penetrating cryoprotectant in which the cells are preserved at a temperature of above 0° C.

Disclosed are kits comprising a storage solution comprising a GAG, such as hyaluronan, and cells wherein the cells are non-cultured.

Disclosed are kits comprising a storage solution comprising a GAG, such as hyaluronan, and cells wherein the cells are stored at a temperature above 0° C.

Disclosed are compositions for preserving cells in the absence of serum comprising cells and a GAG, such as hyaluronan, in which the cells are preserved at a temperature of above 0° C. Also disclosed are compositions for preserving cells in the absence of serum comprising non-cultured cells and a GAG, such as hyaluronan,.

Disclosed are compositions for preserving cells in the absence of serum comprising cells and a GAG, such as hyaluronan, in which the cells are preserved at a temperature of above 0° C. Also disclosed are compositions for preserving cells in the absence of serum comprising cells and a GAG, such as hyaluronan, in the absence of a non cell-penetrating organic solute.

Disclosed are compositions for preserving cells in the absence of serum comprising cells and a GAG, such as hyaluronan, in which the cells are preserved at a temperature of above 0° C. Disclosed are compositions for preserving cells in the absence of serum comprising cells and a GAG, such as hyaluronan, in the absence of a cell-penetrating organic solute

Disclosed are compositions for preserving cells in the absence of serum comprising non-corneal cells and a GAG, such as hyaluronan.

Disclosed are compositions for preserving cells in the absence of serum comprising non-ovarian cells and a GAG, such as hyaluronan.

### a) GAGs

Disclosed herein is a composition comprising a glucosaminoglycan (GAG). Glycosaminoglycans (GAGs) are a class of biocompatible polymers. There are many different types of GAGs, having commonly understood structures, which, for example, are within the disclosed compositions, such as chondroitin sulfate, hyaluronan, dermatan, heparan, heparin, dermatan sulfate, and heparan sulfate. Any GAG known in the art can be used in any of the composites described herein. Glycosaminoglycans can be purchased from Sigma, and many other biochemical suppliers. Alginic acid, pectin, and carboxymethylcellulose are among other carboxylic acid containing polysaccharides useful in the compositions described herein.

The GAGs can be used at concentrations of at least 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, 90%, 95%, 99% or greater by weight to volume.

The GAGs can also be any size, such as high molecular weight preparations or low molecular weight preparations. For example, the GAGs can be preparations having an average size of 1 kDa, 2 kDa, 3 kDa, 4 kDa, 5 kDa, 6 kDa, 7 kDa, 8 kDa, 9 kDa, 10, kDa, 11 kDa, 12 kDa, 13 kDa, 14 kDa, 15 kDa, 16 kDa, 17 kDa, 18 kDa, 19 kDa, 20, kDa, 21 kDa, 22 kDa, 23 kDa, 24 kDa, 25 kDa, 26 kDa, 27 kDa, 28 kDa, 29 kDa, 30, kDa, 31 kDa, 32 kDa, 33 kDa, 34 kDa, 35 kDa, 36 kDa, 37 kDa, 38 kDa, 39 kDa, 40, kDa, 41 kDa, 42 kDa, 43 kDa, 44 kDa, 45 kDa, 46 kDa, 47 kDa, 48 kDa, 49 kDa, 50, kDa, 51 kDa, 52 kDa, 53 kDa, 54 kDa, 55 kDa, 56 kDa, 57 kDa, 58 kDa, 59 kDa, 60, kDa, 61 kDa, 62 kDa, 63 kDa, 64 kDa, 65 kDa, 66 kDa, 67 kDa, 68 kDa, 69 kDa, 70, kDa, 71 kDa, 72 kDa, 73 kDa, 74 kDa, 75 kDa, 76 kDa, 77 kDa, 78 kDa, 79 kDa, 80, kDa, 81 kDa, 82 kDa, 83 kDa, 84 kDa, 85 kDa, 86 kDa, 87 kDa, 88 kDa, 89 kDa, 90, kDa, 91 kDa, 92 kDa, 93 kDa, 94 kDa, 95 kDa, 96 kDa, 97 kDa, 98 kDa, 99 kDa, 100, kDa, 101 kDa, 102 kDa, 103 kDa, 104 kDa, 105 kDa, 106 kDa, 107 kDa, 108 kDa, 109 kDa, 110, kDa, 111 kDa, 112 kDa,113 kDa, 114 kDa, 115 kDa, 116 kDa, 117 kDa, 118 kDa, 119 kDa, 120, kDa, 121 kDa, 122 kDa, 123 kDa, 124 kDa, 125 kDa, 126 kDa, 127 kDa, 128 kDa, 129 kDa, 130, kDa, 131 kDa, 132 kDa, 133 kDa, 134 kDa, 135 kDa, 136 kDa, 137 kDa, 138 kDa, 139 kDa, 140, kDa, 141 kDa, 142 kDa, 143 kDa, 144 kDa, 145 kDa, 146 kDa, 147 kDa, 148 kDa, 149 kDa, 150, kDa, 151 kDa, 152 kDa, 153 kDa, 154 kDa, 155 kDa, 156 kDa, 157 kDa, 158 kDa, 159 kDa, 160, kDa, 161 kDa, 162 kDa, 163 kDa, 164 kDa, 165 kDa, 166 kDa, 167 kDa, 168 kDa, 169 kDa, 170, kDa, 171 kDa, 172 kDa, 173 kDa, 174 kDa, 175 kDa, 176 kDa, 177 kDa, 178 kDa, 179 kDa, 180, kDa, 181 kDa, 182 kDa, 183 kDa, 184 kDa, 185 kDa, 186 kDa, 187 kDa, 188 kDa, 189 kDa, 190, kDa, 191 kDa, 192 kDa, 193 kDa, 194 kDa, 195 kDa, 196 kDa, 197 kDa, 198 kDa, 199 kDa, 200, kDa, 210 kDa, 220 kDa, 230 kDa, 240 kDa, 250 kDa, 260 kDa, 270 kDa, 280 kDa, 290 kDa, 300, kDa 310, kDa, 310 kDa, 320 kDa, 330 kDa, 340 kDa, 350 kDa, 360 kDa, 370 kDa, 380 kDa, 390 kDa, 400, kDa, 410, kDa, 420 kDa, 430 kDa, 440 kDa, 450 kDa, 460 kDa, 470 kDa, 480 kDa, 490 kDa, 500, kDa, 550, kDa, 600 kDa, 650 kDa, 700 kDa, 750 kDa, 800 kDa, 850 kDa, 900 kDa, 950 kDa, 1000 kDa, 1100, kDa, 1200 kDa, 1300, kDa, 1400 kDa, 1500, kDa, 1600 kDa, 1700, kDa, 1800 kDa, 1900, kDa, 2000 kDa, 2100, kDa, 2200 kDa, 2300, kDa, 2400 kDa, 2500, kDa, 2600 kDa, 2700, kDa, 2800 kDa, 2900, kDa, 3000 kDa, 3500, kDa, 4000 kDa, 4500, kDa, 5000 kDa, 5500, kDa, 6000 kDa, 6500, kDa, 7000 kDa, 7500, kDa, 8000 kDa, 8500, kDa, 9000 kDa, 9500, kDa, or 10000 kDa.

For some high molecular weight GAGs it is often in the range of 100 to 10,000 disaccharide units. In another aspect, the lower limit of the molecular weight of the GAG is from 10,000, 20,000, 30,000, 40,000, 50,000, 60,000, 70,000, 80,000, 90,000, or 100,000, and the upper limit is 200,000, 300,000, 400,000, 500,000, 600,000, 700,000, 800,000, 900,000, or 1,000,000, where any of the lower limits can be combined with any of the upper limits.

The amount of GAG used can vary. For example, the molecular weight of the GAG can be inversely proportional to the weight/volume used. In general, the w/v of GAG can be less than 1%, 2%, 3%, 4%, 5%, 10%, 20%, 30%, 40%, 50%, 60%, 70%, 80%, 90%, or less than or equal to 100%.

### (1) Hyaluronic Acid

Hyaluronic acid (HA), which is a member of the GAG family, is a naturally-occurring biopolymer composed of repeating disaccharide units of N-acetyl-D-glucosamine (GlcNAc) D-glucuronic acid (GlcUA) found in the extracellular matrix of all higher animals. For a discussions of HA and GAGs see for example Laurent et al., 18 Acta Chem Scand 274 (1964), Yui et al., 22 J Controlled Rel. 105 (1992), Tomihata and Ikada, 18 Biomaterials 189 (1997), Shah and Barnett, 480 ACS Syinposium Series 116(1991), Larsenetal., In Cosmetic and Pharmaceutical Applications of Polymers C. G. Gebelein, Ed.; Plenum Press: New York, 147 (1991), Kuo et al., 2 Bioconjugates Chem 232 (1991), Pouyani et al., 116 J Am Chem Soc 7515 (1994), Vercruysse et al., 8 Bioconjugate Chem 686 (1997), U.S. Pat. Nos. 4,582,865, 4,713,448, 5,616,568, 5,652,347, and 5,874,417, European Patent Application 0216453 which are herein incorporated by reference at least for their material related to GAGs and HA.

Methods of preparing commercially available hyaluronan and salts thereof are well known. Hyaluronan can be purchased from Seikagaku Company, Clear Solutions Biotech, Inc., Pharmacia Inc., Sigma Inc., and many other suppliers. For high molecular weight hyaluronan it is often in the range of 100 to 10,000 disaccharide units. In another aspect, the lower limit of the molecular weight of the hyaluronan is from 10,000, 20,000, 30,000, 40,000, 50,000, 60,000, 70,000, 80,000, 90,000, or 100,000, and the upper limit is 200,000, 300,000, 400,000, 500,000, 600,000, 700,000, 800,000, 900,000, or 1,000,000, where any of the lower limits can be combined with any of the upper limits.

HA forms highly viscous aqueous solutions, and it takes on an expanded random coil structure due to strong hydrogen bonding. The coiled structure allows it to trap approximately 1000 times its weight in water. HA has unique physiochemical properties as well as distinctive biological functions. These functions, relationships, and interactions are discussed for example in Laurent, T. C., Laurent, U. B. G., and Fraser, J. R. E. (1995) Functions of hyaluronan. Ann Rheum Dis 54, 429-432; Fraser, J. R. E., Laurent, T. C., and Laurent, U. B. G. (1997) Hyaluronan: Its nature, distribution, functions and turnover. J Intern Med 242, 27-33; Dowthwaite, G. P., Edwards, J. C. W., and Pitsillides, A. A. (1998) An essential role for the interaction between hyaluronan and hyaluronan binding proteins during joint development. J Histochem Cytochem 46, 641-651; Collis, L., Hall, C., Lange, L., Ziebell, M. R., Prestwich, G. D., and Turley, E. A. (1998) Rapid hyaluronan uptake is associated with enhanced motility: implications for an intracellular mode of action. FEBS Lett. 440, 444-449; Hardwick, C., Hoare, K., Owens, R., Hohn, H. P., Hook, M., Moore, D., Cripps, V., Austen, L., Nance, D. M., and Turley, E. A. (1992) Molecular cloning of a novel hyaluronan receptor that mediates tumor cell motility. J. Cell Biol. 117, 1343-1350; Entwistle, J., Hall, C. L., and Turley, E. A. (1996) Receptors: regulators of signalling to the cytoskeleton. J Cell Biochem 61, 569-577; and Cheung, W. F., Cruz, T. F., and Turley, E. A. (1999) Receptor for hyaluronan-mediated motility (RHAMM), a hyaladherin that regulates cell responses to growth factors. Biochem. Soc. Trans. 27, 135-142; Toole, B. P. (1997) Hyaluronan in morphogenesis. J Intern Med 242, 35-40; and Kim et al., 9 Pharm Res 283, (1992), which are herein incorporated by reference at least for their material related to GAGs and HA and their function and properties.

### (2) Chondroitin Sulfate

Another member of the GAG family is chondroitin sulfate (CS). CS is comprised of alternating units of β-1,3-linked glucuronic acid and (β-1,4). N-acetyl-galactosamine (GalNAc) and is sulfated on the 4- or 6- position of the GalNAc residues. CS is usually found bound to a core protein forming a proteoglycan, e.g. aggrecan or versican. Aggregan is the primary proteoglycan in cartilage, and its primary function is to swell and hydrate the collagen fibril framework. Versican is believed to play a role in intracellular signaling, cell recognition, and connecting ECM components to cell surface glycoproteins. Additionally, CS proteoglycans like neurocan and phosphacan play important roles in axon growth and pathfinding.

### (3) Chemically Modified GAGs

Recently, GAG molecules have been chemically modified (Luo, Y., Kirker, K. R., and Prestwich, G. D. (2000) Cross-linked hyaluronic acid hydrogel films: new biomaterials for drug delivery (Journal of Controlled Release 69, 169-184; Pouyani, T., Harbison, G. S., and Prestwich, G. D. (1994) Novel hydrogels of hyaluronic acid: synthesis, surface morphology, and solid-state NMR. J Am Chem Soc 116, 7515-7522; and Pouyani, T., and Prestwich, G. D. (1994) Functionalized derivatives of hyaluronic acid oligosaccharides—drug carriers and novel biomaterials. Bioconjugate Chemistry 5, 339-347 which are herein incorporated by reference in their entireties at least for material related to chemically modified GAGs and HA.).

### b) Media

One particular media that can be used is RPMI-1640. RMPI-1640 is made by Hyclone. RMPI-1640 comprises inorganic salts, ranging in concentrations from 48 to 6000 mg/L, such as RCa(NO3)2·4H2O, KCl, MgSO4 (anhydrous), NaCl, NaH2PO4 (anhydrous), amino acids, ranging in concentrations from 5 to 300 mg/L, such as, L-Arginine HCl, L-Asparagine, L-Aspartic Acid, L-Cystine 2HCl, L-Glutamic Acid, L-Glutamine, Glycine, L-Histidine FB, L-Hydroxyproline, L-Isoleucine, L-Leucine, L-Lysine HCl, L-Methionine, L-Phenylalanine, L-Proline, L-Serine, L-Threonine, L-Tryptophan, L-Tryosine 2Na·2H2O, L-Valine, vitamins, ranging in concentrations from 0.0050 to 35 mg/L such as, d-Biotin, D-Ca Pantothenate, Choline Chloride, Folic AcidMyo-Inositol, Niacinamide, Pyridoxine HCl, Riboflavin, Thiamine HCl, Vitamin B-12, and other components, ranging in concentrations from 1 to 5958 mg/L, such as D-Glucose, Para-Aminobenzoic Acid (PABA), Glutathione(Reduced), Phenol Red (Sodium), HEPES, and NaHCO3.

RPMI-1640 is an example of a media that can be used. Furthermore, it is an example of the types of components that can go into a media. It is not required that each of these components be present, but nor is it required that the components be in a particular concentration. It is understood that each combination of individual components listed for the inorganic salts, amino acids, vitamins and other components described for RPMI-1640 is described considered individually described herein. It also is understood that each individual concentration between the concentrations listed for the inorganic salts, amino acids, vitamins and other components described for RPMI-1640 is considered individually described herein. Furthermore, the optimization of components and/or the concentrations can be accomplished by screening with different combinations or concentrations.

Other medias can include Basal Medium Eagle (BME), Dulbecco's Modified Eagles Medium (DME), Nutrient Mixture Ham's F-10, Nutrient Mixture Ham's F-12, Dulbecco's Modified Eagles Medium Nutrient Mixture F-12 Ham (DME/F12 1:1 mixture), L-15 Medium Leibovitz, McCoy's 5A Medium, Medium 199, Minimum Essential Medium Eagle, RPMI-1640 Medium, or Waymouth's Medium. More specifically, the preservation compositionmedium can further comprise from about 5-20% Fetal Bovine Serum. Typically cells which are not cryopreserved can include placing cells or tissues to be cultured in a medium comprising a GAG and less than or equal to 95% of the medias disclosed herein.

### c) Other Materials Allowed in Solution

Compositions useful with the storage media described herein include, but are not limited to, an extracellular matrix protein, a chemically-modified extracellular matrix protein, or a partially hydrolyzed derivative of an extracellular matrix protein. The proteins may be naturally occurring or recombinant polypeptides possessing a cell interactive domain. The protein can also be mixtures of proteins, where one or more of the proteins are modified. Specific examples of proteins include, but are not limited to, collagen, elastin, decorin, laminin, or fibronectin.

In one aspect, the storage media includes cross-linked alginates, gelatin, collagen, cross-linked collagen, collagen derivatives, such as, succinylated collagen or methylated collagen, cross-linked hyaluronan, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyoxanones and polyoxalates, copolymer of poly(bis(p-carboxyphenoxy)propane)anhydride (PCPP) and sebacic acid, poly(1-glutamic acid), poly(d-glutamic acid), polyacrylic acid, poly(d1-glutamic acid), poly(1-aspartic acid), poly(d-aspartic acid), poly(d1-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin. In another aspect, highly cross-linked HA can be the prohealing compound.

The storage media can optionally contain a second compound. In one aspect, the second compound can be a growth factor. Any substance or metabolic precursor which is capable of promoting growth and survival of cells and tissues or augmenting the functioning of cells is useful as a growth factor. Examples of growth factors include, but are not limited to, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), bone morphogeneic proteins (BMPs), dried bone material, and the like; and antineoplastic agents such as methotrexate, 5-fluorouracil, adriamycin, vinblastine, cisplatin, tumor-specific antibodies conjugated to toxins, tumor necrosis factor, and the like. The amount of growth factor incorporated into the media will vary depending upon the growth factor and prohealing compound selected as well as the intended end-use of the cell, tissue, or organ.

Any of the growth factors disclosed in U.S. Pat. No. 6,534,591 B2, which is incorporated by reference in its entirety, can be used in this aspect. In one aspect, the growth factor includes transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifuictional regulatory proteins. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

In one aspect, any of the storage media described above can include at least one pharmaceutically-acceptable compound. The resulting pharmaceutical composition can provide a system for sustained, continuous delivery of drugs and other biologically-active agents to those cells, such as tissues and organs, being stored. The biologically-active agent is capable of providing a local or systemic biological, physiological or therapeutic effect in the biological system to which it is applied. For example, the agent can act to control infection or inflammation, enhance cell growth and tissue regeneration, control tumor growth, act as an analgesic, promote anti-cell attachment, and enhance bone growth, among other functions.

In one aspect, the pharmaceutically-acceptable compounds can include substances capable of preventing an infection systemically in the biological system or locally in the cells being stored or at the defect site after transplantation has taken place, as for example, anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. All compounds are available from Sigma Chemical Co. (Milwaukee, Wis.).

The preservation compositions can comprise at least 100 Units/milliliter penicillin, at least 0.1 milligrams/milliliter streptomycin, and at least 0.25 micrograms/milliliter amphotericin B. The medium may comprise about 10% Fetal Bovine Serum, at least 100 Units/milliliter penicillin, at least 0.1 milligrams/milliliter streptomycin, and at least 0.25 micrograms/milliliter amphotericin B. The media can contain other antimicrobials including silver and any effective microbial agent at a concentration determined to have efficacy.

In another embodiment, a process for tissue decontamination can be included in the preserving of viable tissues and cells comprising: incubating tissues in an about 1:30 chlorhexidine solution for not more than about 3 minutes; agitating tissues during the incubation period; placing tissues into a medium comprising Ham's F-12 and at least 600 Units/milliliter penicillin, at least 5 milligrams/milliliter streptomycin, and at least 1.5 micrograms/milliliter amphotericin B.; soaking tissues for about 40 minutes at about 4° C.; and proceeding with tissue isolation procedures, for example, as described herein.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaprotemal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All compounds are available from Sigma Chemical Co. (Milwaukee, Wis.).

### (1) Cryopresevants

“Cryoprotectant” refers to chemical compounds which are added to biological samples in order to minimize the deleterious effects of cryopreservation procedures.

When the cells of the tissue are preserved at temperatures below freezing, they are said to be “cryopreserved.” When talking about cryopreservation the preservation is referred to in as cells, but it is understood that this applicable for tissues that the cells can make up as well. When employing freezing techniques to preserve biological materials, high concentrations (approximately 10% by volume) of cryoprotectants, such as glycerol, dimethylsulfoxide (DMSO), glycols or propanediol, are often introduced to the material prior to freezing in order to limit the amount of damage caused to cells by the formation of ice crystals during freezing.

In the method of cryopreservation, the cells are protected during cryopreservation by being brought into contact with a cryopreservation composition prior to freezing to the cryopreservation temperature. By being brought into contact with the cryopreservation composition is meant that the cells are made to be in contact in some manner with the cryopreservation composition so that during the reduction of temperature to the cryopreservation temperature, the cells are protected by the cryopreservation composition. For example, the cells may be brought into contact with the cryopreservation composition by filling the appropriate wells of a plate to which the cells to be protected are attached, by suspending the cells in a solution of the cryopreservation composition, etc.

The cells to be cryopreserved can also be in contact with a freezing compatible pH buffer comprised most typically of at least a basic salt solution, an energy source (for example, glucose) and a buffer capable of maintaining a neutral pH at cooled temperatures. Well known such materials include, for example, Dulbecco's Modified Eagle Medium (DMEM). This material may also be included as part of the cryopreservation composition.

The cryopreservation composition may comprise any cryoprotective materials known in the art without limitation. The cryopreservation composition can be used before, after, or during treatment with the GAG. For example, cells can be stored at 4° C. in a solution containing a GAG, and then cryopreserved. Known cryoprotectant compounds include, for example, but are not limited to, acetamide, agarose, alginate, 1-analine, albumin, ammonium acetate, butanediol, chondroitin sulfate, chloroform, choline, dextrans, diethylene glycol, dimethyl acetamide, dimethyl formamide, dimethyl sulfoxide (DMSO), erythritol, ethanol, ethylene glycol, formamide, glucose, glycerol, .alpha.-glycerophosphate, glycerol monoacetate, glycine, hydroxyethyl starch, inositol, lactose, magnesium chloride, magnesium sulfate, maltose, mannitol, mannose, methanol, methyl acetamide, methylformamide, methyl ureas, phenol, pluronic polyols, polyethylene glycol, polyvinylpyrrolidone, proline, propylene glycol, pyridine N-oxide, ribose, serine, sodium bromide, sodium chloride, sodium iodide, sodium nitrate, sodium sulfate, sorbitol, sucrose, trehalose, triethylene glycol, trimethylamine acetate, urea, valine, xylose, etc. The cryoprotectant compounds are preferably present in the cryopreservation composition in an amount of from, for example, 0.05 M to 6.0 M, preferably 0.1 to 3.0 M.

The rate of change from room temperature to 1-2° C. below the freezing point of the solution may have a major effect on ultimate viability if the cells are sensitive to thermal shock. Between 3.5° C. and −5° C., the sample is normally induced to freeze either by the introduction of an ice crystal, by touching the surface of the media with a cold probe, by mechanical vibration, or by rapidly lowering the temperature until ice nucleation occurs. Since freezing is an exothermic process, heat must be conducted away from the freezing solution. This may be done either by keeping the samples immersed in a liquid with a low freezing point or by providing a substantial heat sink. As ice forms in the extracellular media, more and more free water becomes bound in the ice phase. Cell membranes, being hydrophobic, act as a barrier for the nucleation of intracellular ice and therefore unfrozen cells are exposed to an increasingly hypertonic solution. The extracellular salt concentration increases as a consequence of water sequestration into ice. The unfrozen cells shrink due to the transport of water out of the cell in response to the osmotic imbalance between the intracellular and extracellular fluid phases. The sample is then cooled at a finite rate which must be optimized for each cell type.

The optimal rate of cooling is determined by the permeability of the cell membrane to water, the surface-to-volume ratio of the cell, along the type and concentration of cryoprotective additives. For most nucleated mammalian cells frozen in glycerol or DMSO, the optimal cooling rate usually is between about 0.3° to 10° C. per minute. Continuous cooling between about 4° C. and −80° C. is the most commonly used. Once the sample reaches approximately −80° C., it can be transferred directly into liquid nitrogen (−196° C.) or into the vapor phase of liquid nitrogen for storage.

The duration of viable cell storage at liquid nitrogen temperature is dependent predominantly on the rate of generation of free radicals caused by the cosmic ray background. For example, the half-life for mammalian embryos stored in liquid nitrogen has been estimated to be approximately 30,000 years. It is important not to allow frozen cells to warm above their storage temperature for even brief periods of time. Intermittent warming promotes rapid migratory recrystallization, which can damage cellular structure and decrease overall viability.

The optimal rate of thawing of the sample is dependent on the freezing conditions used. In general, for single cells frozen in suspension, and for tissues such as heart valves, a rapid rate of warming is desirable. Such rapid warming limits the growth of ice crystals in the frozen samples and is often an absolute requirement for high survival. With many tissues this warming can be accomplished by agitating the sample in a 37-42° C. water bath. The rationale for rapid warming is that it limits the growth of ice crystals which were formed during cooling. Some tissues may be sensitive to rapid warming. This is due to transient osmotic shock, because the cells are exposed to an extracellular hypertonic solution as the ice melts and are forced to rehydrate in order to maintain their osmotic equilibrium. For other, more sensitive, samples, metabolic processes can be reactivated or brought up to normal levels by successive dilutions using serum or other high molecular weight polymers in the thawing medium.

Upon completion of the thawing procedure, the cells are still exposed to multimolar concentrations of cryoprotective agents which must be gradually diluted to return the cells to an isotonic media. The cells can be exposed to GAGs at this point, for example, in the amounts disclosed above. This also reduces dilution induced osmotic shock. For mammalian cells, a stepwise dilution protocol is typically used. The dilution of the sample is normally carried out at preferably 4° C., so as to reduce the effects of both osmotic shock and cryoprotectant toxicity. When glycerol is used, care must be taken to insure complete mixing of the physiological salt solution with the cryoprotective solution. Overly rapid dilution can result in the cells being exposed to a potentially damaging osmotic stress while very slow dilution may result in toxicity to the cells from prolonged exposure to the cryoprotective agent(s).

The cells can also be preserved at a temperature above freezing in a solution containing a GAG. Typically, cells can be stored at 4° C. for several days or weeks and maintain viability.

Other additives can include antioxidants, including vitamin E, C, chelators and anti-apoptotic agents, for example.

### 2. Cells, Tissues, and Organs

The methods and compositons disclosed herein are useful with a wide array of cells, tissues, and organs. For example, any type of cell can be used, including totipotent, embryonic, and somatic.

A “precursor cell” can be any cell in a cell differentiation pathway that is capable of differentiating into a more mature cell. As such, the term “precursor cell population” refers to a group of cells capable of developing into a more mature cell. A precursor cell population can comprise cells that are totipotent, cells that are pluripotent and cells that are stem cell lineage restricted (i.e. cells capable of developing into less than all hematopoietic lineages, or into, for example, only cells of erythroid lineage). As used herein, the term “totipotent cell” refers to a cell capable of developing into all lineages of cells. The first few cell divisions in embryonic development produce totipotent cells. After four days of embryonic development, the cells begin to specialize into pluripotent stem cells. Also as used herein, the term “pluripotent cell” refers to a cell capable of developing into a variety of lineages and are at least able to develop into all hematopoietic lineages (e.g., lymphoid, erythroid, and thrombocytic lineages).

Pluripotent cells undergo further specialization into multipotent cells that are commited to give rise to cells that have a particular function. Multipotent stem cells give rise to a limited range of cells within tissue types. The offspring of the pluripotent cells become the progenitors of such cell lines as blood cells, skin cells, and nerve cells. At this stage, they are multipotent. For example, multipotent blood stem cells give rise to the red cells, white cells, and platelets of blood.

A “pluripotent population of cells” refers to a composition of cells capable of developing into less than all lineages of cells but at least into all hematopoietic lineages. As used herein, the terms “develop”, “differentiate” and “mature” all refer to the progression of a cell from the stage of having the potential to differentiate into at least two different cellular lineages to becoming a specialized cell. Such terms can be used interchangeably.

Any of a wide range of somatic cells, including adult derived stem cells from various tissues, can be cryopreserved using the compositions and methods disclosed herein. As used herein, the term “somatic cell” refers to any cell that is not a gamete (sperm or oocyte) or a totipotent cell. Exemplary somatic cells which can be cryopreserved include, for example, epithelial, connective tissue, muscle, amniocyte, nerve, brain, mucosal, blood, cartilage, mammary, kidney, liver, pancreatic, bone, corneal, arterial, lung, and skin cells. Somatic cells derived, for example, from the circulatory system can be cryopreserved. Mammalian cells including porcine, canine, human, murine, equine and bovine cells can be cryopreserved. In another embodiment, somatic avian cells, tumor cells, or genetically altered cells may be cryopreserved using the compositions disclosed herein.

The composition comprising a GAG can be for preserving cells. The cells can be non-cultured or cultured. The cells can be preserved at a temperature of above freezing or a temperature below freezing. The cells can be unkeratinized epithelial cells, but do not need to be unkeratinized epithelial cells. The cells can be corneal or non-corneal, ovarian or non-ovarian, and musculoskeletal or non-musculoskeletal. The cells can be in the absence of a non-cell penetrating cryoprotectant, such as algae-derived polysaccharides, which are a class of nonpermeating cryoprotectants. These naturally occurring polysaccharides are known as agaroses and alginates. Storage of organs, such as heart and kidneys, at temperatures below 0° C. frequently results in the loss of many cells with a corresponding reduction in viability of the organ. Such complex biological materials can therefore be stored in aqueous, saline-based media at temperatures above freezing, typically around 4° C. Saline-based media typically consist of isotonic saline (sodium chloride 0.154 M) which has been modified by the addition of low concentrations of various inorganic ions, such as sodium, potassium, calcium, magnesium, chloride, phosphate and bicarbonate, to mimic the extracellular environment. Small amounts of compounds such as glucose, lactose, amino acids and vitamins are often added as metabolites. All saline-based media used for preservation of biological materials have high electrical conductivity. Examples of media currently employed for the preservation of biological materials include phosphate-buffered saline (PBS), M-2 (a Hepes buffered murine culture medium), Ringer's solution and Krebs bicarbonate-buffered medium. University of Wisconsin (UW) Solution can also be used for organ storage.

The cells disclosed herein can be in sheets, such as a cell layer, or can occur separately, such as a colony. The cells can comprise a tissue, or an organ, and can be stored in solution or not in a solution. As disclosed above, the cells can be engineered tissue constructs containing cells. These include cells, including stem cells, which are grown in an engineered matrix for eventual transplantation to a recipient.

“Tissue” means a collection of similar cells and the intercellular substances surrounding them. There are four basic tissues in the human body: (1) epithelium; (2) connective tissues, including blood, bone and cartilage; (3) muscle tissue; and (4) nerve tissue. It is understood that tissues are made up of one or more types of cells, and that each of these cells or cell types can also be preserved individually or in combination, but that the cell or cell type may not be considered a tissue. Included in the definition of “tissue” are engineered tissue constructs containing cells. These include cells, including stem cells, which are grown in an engineered matrix for eventual transplantation to a recipient.

The tissues of the present invention include graft cells. By “graft cells” is meant those cells, tissues or organs obtained from a donor for transplantation into a recipient where the graft cells may be derived from human subjects or from animals and may be transplanted from one subject back into the same subject or from one subject (the donor) into another subject (the recipient) for the purpose of improving the health of the recipient or for research purposes. The donor subject can be a living subject, fetus, or a recently dead subject. The grafts include replenishable cells taken from a healthy donor such as stem cells, blood cells, bone marrow cells, placental cells, liver cells, sperm, and ova. Also included are organs removed from a healthy donor such as the kidney as well as organs removed from a cadaver at point of death including heart, lungs, liver, pancreas and corneal tissue. This last group includes fetal tissue such as brain tissue taken from an aborted fetus.

Tissues that are protected by the method of the invention may be derived from animals or humans, children, adult or fetal tissue and include, but are not limited to, blood and all of its components, including erythrocytes, leukocytes, platelets, serum, central nervous tissue, including brain and spinal cord tissue, neurons, and glia; peripheral nervous tissue, including ganglia, posterior pituitary gland, adrenal medulla, and pineal; connective tissue, including skin, ligaments, tendons, and fibroblasts; muscle tissue, including skeletal, smooth and cardiac tissues or the cells therefrom; endocrine tissue, including anterior pituitary gland, thyroid gland, parathyroid gland, adrenal cortex, pancreas and its subparts, testes, ovaries, placenta, and the endocrine cells that are a part of each of these tissues; blood vessels, including arteries, veins, capillaries and the cells from these vessels, lung tissue; heart tissue and whole organ; heart valves; liver; kidney; intestines; bone; immune tissue, including blood cells, bone marrow and spleen; eyes and their parts; reproductive tract tissues; and urinary tract tissue.

The methods and compositions disclosed herein can be applied to blood transfusions in which erythrocytes are transferred from an animal donor back to the donor or to an animal recipient or archived indefinitely, storage and protection of a tissue or tissue type during transplantation, for example, fetal tissue for fetal brain transplants in the treatment of Parkinson's disease, skin transplantation for burn victims, the heart during transplantation, and body parts for reattachment after accidental severance.

Also disclosed is the storage of organs. Such organs include, but are not limited to liver, pancreas, lung, kidney, skin, eyes, heart, or any other transplantable organ of the body or part thereof.

The donor and/or recipient of the cells, tissue, and/or organ protected by this invention is not species restricted but may be applied to tissue from any animal, including mammals, such as domestic animals, for example, pigs, cows and sheep, and primates as well as humans. This invention is directed to transplantation of tissue from one member to another of the same species or back to the same individual as well as from a member of one species to a member of another species.

### 3. Time

The viability of biological materials stored in saline-based media gradually decreases over time. Loss of viability is believed to be due to the build-up of toxic wastes, and loss of metabolites and other supporting compounds caused by continued metabolic activity. Using conventional saline-based media, living tissues can only be successfully preserved for relatively short periods of time. Examination of the microstructure of organs stored towards the upper limit of time shows degeneration, such as of mitochondria in heart muscle, and the performance of the organ once replaced is measurably compromised. For example, a human heart can only be stored in cold ionic solutions for about 5 hours following removal from a donor, thereby severely limiting the distance over which the heart can be transported.

The disclosed compositions allow for the liquid storage of tissues for days or longer, without losing the properties of the tissue. The loss of properties of the tissue refers to all of the properties for that particular tissue that make the tissue useful, and their loss is defined as the state where a sufficient amount of the properties is no longer present, such that the tissue is no longer suitable for transplantation. The disclosed compositions can prevent the loss of the tissue properties for up to 25 days or longer, for example. One way of calculating the loss of the cell or tissue properties is to determine the percentage of viable cells or tissues that remain after a certain point in time. Traditionally, in liquid media, non-cryo preservation situations the amount of viable cells or tissue, cells or tissue that can be successfully transplanted, decreases overtime. Thus, for example, disclosed are compositions where at least 50%, 55%, 60%, 65%, 70%, 75%, 80%, 85%, 90%, 95%, or 99% of the cells are viable after 10 days. It is understood that this type of assay can be performed at any day length disclosed herein, and at any % of viable cells. Tetrazolium reduction (WST-1) assay for cell proliferation and viability assessment can be used. 5-Bromo-2′-deoxy-uridine Labeling and Detection Kit to measure DNA synthesis or cell proliferation in the cells or tissue is another option, for example.

The compositions are useful for extending the length of time at non-freezing temperatures tissues can be stored. The compositions can be used at a variety of temperatures including 0, 1, 2, 3, 4, 5, 6, 7, 8, 9, 10, 11, 12, 25, 37, 42, 45, or 60 degrees Celsius, or any point in between.

### C. Methods

Also disclosed is a method of making a cell-containing storage solution comprising utilizing a solution comprising a glucosaminoglycan, and placing the cells in the solution. The solution can contain serum, although this is not necessary. The storage solution can also comprise any of the cryoprotectants or other agents described herein.

Also disclosed is a method of treatment comprising obtaining cells; storing the cells in a solution comprising glucosaminoglycan in the absence of serum; and using the cells in treatment. The cells can be obtained from any source and can be used in the treatment of any disease for which transplantation may be beneficial. The cells can also be used in research.

The methods can include the preserving any type of tissue in the compositions disclosed herein, in the manner disclosed herein, with the characteristics disclosed herein. The composites and pharmaceutical compositions described herein can be used for a variety of uses related to wound healing, burn injury healing, organ transplantation, and tissue regeneration.

It is understood that the disclosed composites and compositions can be applied to a subject in need of tissue regeneration. For example, cells stored in the storage media can be used as described herein for implantation. Examples of subjects that can be treated with the composites described herein include mammals such as mice, rats, cows or cattle, horses, sheep, goats, cats, dogs, and primates, including apes, chimpanzees, orangatangs, and humans. In another aspect, the composites and compositions described herein can be applied to birds.

When being used in areas related to tissue regeneration such as wound or burn healing, it is not necessary that the disclosed compositions and methods eliminate the need for one or more related accepted therapies. It is understood that any decrease in the length of time for recovery or increase in the quality of the recovery obtained by the recipient of the disclosed compositions and methods has obtained some benefit.

### D. Kits

Also disclosed are kits. For example, a kit can comprise a storage solution comprising a GAG. The storage solution can contain any of the components listed herein, or can comprise any standard solution known to those in the art. For example, the storage solution can comprise saline or serum. The GAG can be any of those GAGs disclosed herein, such as, for example, HA or chondroitin sulfate. The storage solution can be for storage of cells above freezing, for storage of cells at freezing, or for the storage of cells below freezing, such as in cryopreservation.

It is understood that any given particular aspect of the disclosed compositions and methods can be easily compared to the specific examples and embodiments disclosed herein. By performing such a comparison, the relative efficacy of each particular embodiment can be easily determined. Particularly preferred assays for the various uses are those assays which are disclosed in the Examples herein, and it is understood that these assays, while not necessarily limiting, can be performed with any of the composites, compositions, and methods disclosed herein.

### E. EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### 1. Example 1

Preparation of Media with Various Sizes and Concentrations of Hyaluronan.

Glycosaminoglycans (GAGs), including hyaluronan (HA) and chondroitin sulfate (CS), are aminosugar-containing polysaccharides in the extracellular matrix (ECM) of all vertebrates. HA is the only non-sulfated GAG and is comprised of alternating units of -1,4-linked D glucuronic acid and (β-1,3) N-acetyl-D-glucosamine. HA is non-immunogenic and forms highly viscous aqueous solutions, endowing HA with unique physicochemical properties as well as distinctive biological functions (Fraser J R E, Laurent T C, and Laurent U B G. Hyaluronan: Its nature, distribution, functions and turnover. J. Intern. Med. 1997;242(1):27-33), including the maintenance of extracellular space and the transport of ion solutes and nutrients, regulation of cell adhesion and motility (Collis L, Hall C, Lange L, Ziebell M R, Prestwich G D, and Turley E A. Rapid hyaluronan uptake is associated with enhanced motility: implications for an intracellular mode of action. FEBS Lett. 1998;440(3):444-449; Hardwick C, Hoare K, Owens R, Hohn H P, Hook M, Moore D, Cripps V, Austen L, Nance D M, and Turley E A. Molecular cloning of a novel hyaluronan receptor that mediates tumor cell motility. J. Cell Biol. 1992;117:1343-1350), cell proliferation and differentiation (Gerdin B and Hallgren R. Dynamic role of hyaluronan (HYA) in connective tissue activation and inflammation. J. Intern. Med. 1997;242(1):49-55), modulation of inflammation (Boyce D E, Thomas J H, Moore K, and Harding K. Hyaluronic acid induces tumour necrosis factor-β production by human macrophages in vitro. British J. Plastic Surgery 1997;50:362-368), and angiogenesis and healing (Iocono J A, Krummel T M, Keefer K A, Allison G M, and Paul H. Repeated additions of hyaluronan alters granulation tissue deposition in sponge implants in mice. Wound Repair Regen. 1998;6(5):442-448). Hyaluronan, an effective scavenger of free radicals, can also serve a protective role in epidermis by scavenging reactive oxygen species. The rapid turnover of hyaluronan can help to remove and clear noxious compounds from the epidermis (Moseley R, Leaver M, Walker M, Waddington R J, Parsons D, Chen W Y, Embery G. Comparison of the antioxidant properties of HYAFF-11p75, AQUACEL and hyaluronan towards reactive oxygen species in vitro. 2002 Biomaterials 23:2255-64).

Various sized HA are produced as follows: to produce 200 kDa HA, high molecular weight HA (MW=1.5×10⁶) is degraded under acidic conditions. HA (20 g) is dissolved in 2.5 L H₂O by vortexing at 150 rpm at 37° C. After 3 hr, the solution is moved to a mechanical stirrer and the pH of the solution adjusted to 0.6-0.7 by the addition of concentrated HCl, and stirred for 24 hr. The solution is neutralized with 0.1 N NaOH, and dialyzed (MWCO=3,500) exhaustively against H₂O. The molecular weight of the resulting low molecular weight HA is determined by GPC. The various sized HA are added to RPMI-1640 to be used as storage media for human skin at 4° C.

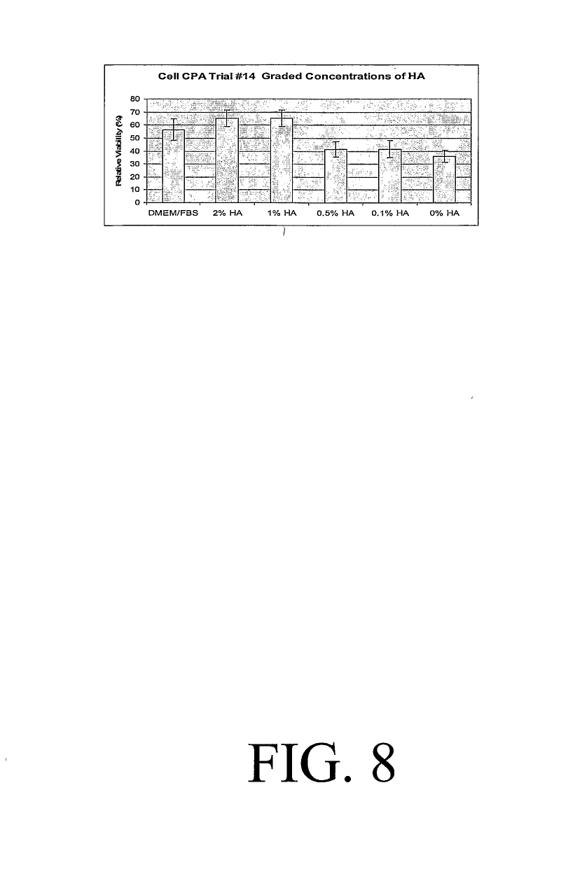

Various concentrations of HA can be used in cryopreservation. For example, FIG. 8 shows that HA at concentrations of 0.1%, 0.5%, 1%, and 2% protect cell viability in post- thaw cells at 90 minutes. The protocol used in this experiment follows. Table 1 shows the results.

### a) Freezing Protocol

Human preadipocytes were harvested from a cadaveric donor. Cells were then cultured in FBS supplemented DMEM until the desired cell number was obtained. The cells were then harvested with 0.025% trypsin, and 1×10⁶ cells used for each experimental solution. The cells were first suspended with ½ mL of desired solution without DMSO, then the cell suspensions were cooled in an ice bath for 5 minutes. 250 uL of cryosolution containing 20% DMSO was added, and the cells returned to an ice bath for 5 minutes. Additional 250 uL of cryosolution with DMSO was added to achieve a total volume of 1 mL with final concentration of DMSO equal to 10%. Cryovials were kept on ice until the cells were cryopreserved with controlled rate of the freezer, then stored in a liquid nitrogen storage tank

### b) Thawing Protocol

Cells were transferred to −20° C. freezer for 30 minutes, then the cyrovials were opened in a clean bench to release pressure. They were then rapidly thawed in 37° C. water bath only until the last ice was melted. They were then cooled in an ice bath for 5 minutes. ½ mL mannitol solution was then added, and the cells were allowed to cool 3 minutes. An additional ½ mL mannitol solution was added, and again the cells were allowed to cool 3 minutes. They were then transferred to a 50 mL conical tube with 2 mL cold DMEM, and returned to an ice bath for 2 minutes. 16 mL of cold DMEM was added and placed in an ice bath for 2 minutes. They were then centrifuged for 5 minutes and the supernatant was aspirated. The cells were then resuspended in 4 mL culture media, and a WST-1 viability assay was performed with 200 uL cell suspension and 20 uL WST-1 reagent.

TABLE 1
Cell CPA Trial
MODE - Single
WAVE LENGTH - 450 nm
DATE - Oct. 05, 2004
TIME - 12:00:38 PM
Pre-freeze WST-1 data for 5 * 10\hat{ }4 cells at 90 minutes

| | | b- | |
|---|---|---|---|
| fresh cells | | ground | |
| 2.767 | 2.598 | 0.355 | 0.352 |
| 2.736 | 2.217 | 0.344 | 0.367 |
| 2.705 | 2.423 | 0.358 | 0.355 |
| Mean | 2.574333 | mean | 0.355167 |
| | adjusted | 2.219167 | |

Post-Thaw WST-1 Data at 90 minutes

| | | | | | | | | | |
|---|---|---|---|---|---|---|---|---|---|
| DMEM/ | 1.566 | 1.982 | 1.958 | 1.677 | 1.613 | 1.561 | 1.42 | 1.568 | 1.456 |
| FBS | | | | | | | | | |
| 2% | 2.025 | 1.79 | 1.84 | 1.97 | 1.773 | 1.622 | 1.698 | 1.851 | 1.919 |
| HA | | | | | | | | | |
| 1% | 1.68 | 1.648 | 1.964 | 1.636 | 1.937 | 2.083 | 1.902 | 1.958 | 1.935 |
| HA | | | | | | | | | |
| 0.5% | 1.33 | 1.326 | 1.56 | 1.107 | 1.61 | 1.349 | 1.273 | 1.315 | 1.34 |
| HA | | | | | | | | | |
| 0.1% | 1.314 | 1.147 | 1.265 | 1.199 | 1.461 | 1.706 | 1.445 | 1.429 | 1.324 |
| HA | | | | | | | | | |
| 0% | 1.272 | 1.082 | 1.113 | 1.171 | 1.248 | 1.336 | 1.385 | 1.067 | 1.182 |
| HA | | | | | | | | | |
| b- | 0.429 | 0.413 | 0.429 | 0.416 | 0.416 | 0.412 | 0.437 | 0.427 | 0.423 |
| ground | | | | | | | | | |

| | mean | S.D. | adj. | viability | S.D. |
|---|---|---|---|---|---|
| DMEM/ | 1.677833 | 0.184151 | 1.251667 | 56.4025535 | 8.298207 |
| FBS | | | | | |
| 2% HA | 1.8735 | 0.142268 | 1.447333 | 65.2196771 | 6.410889 |
| 1% HA | 1.874167 | 0.143794 | 1.448 | 65.2497184 | 6.479637 |
| 0.5% HA | 1.34875 | 0.131507 | 0.922583 | 41.5734134 | 5.925951 |
| 0.1% HA | 1.349167 | 0.146321 | 0.923 | 41.5921893 | 6.593532 |
| 0% HA | 1.225583 | 0.104891 | 0.799417 | 36.023282 | 4.726586 |
| b-ground | 0.426167 | 0.01053 | 0 | | |

TABLE 2
Cell CPA Trial
Pre-freeze WST-1 data for 5 * 10\hat{ }4 cells at 60 minutes
MODE - Single
WAVE LENGTH - 450 nm
INSTRUMENT - THERMOmax
DATE - Oct. 19, 2004
TIME - 1:12:19 PM

| | | | | | | | | mean | S.D. | adjusted |
|---|---|---|---|---|---|---|---|---|---|---|
| 2.004 | 1.51 | 1.516 | 1.416 | 1.512 | 1.508 | 1.483 | 1.851 | 1.6 | 0.208781 | 1.298375 |
| 0.31 | 0.302 | 0.297 | 0.297 | 0.3 | 0.306 | 0.301 | 0.3 | 0.301625 | 0.004438 | |

Post-Thaw WST-1 Data at 60 minutes

| | | | | | | | | | | | | |
|---|---|---|---|---|---|---|---|---|---|---|---|---|
| DMEM/FBS | 1.29 | 1.497 | 1.09 | 1.114 | 1.151 | 1.154 | 1.068 | 1.096 | 0.986 | 1.163 | 1.191 | 1.184 |
| 2% HA | 1.267 | 1.124 | 1.161 | 1.071 | 1.402 | 1.229 | 1.111 | 1.228 | 1.073 | 1.042 | 1.041 | 1.485 |
| 1% HA | 1.062 | 0.912 | 0.863 | 0.882 | 1.085 | 0.975 | 0.982 | 0.809 | 0.642 | 0.789 | 0.654 | 1.06 |
| 0.5% HA | 1.111 | 0.79 | 0.748 | 0.994 | 1.182 | 1.356 | 1.068 | 0.928 | 0.664 | 0.874 | 0.74 | 1.092 |
| 0.1% HA | 1.044 | 0.979 | 0.889 | 0.876 | 0.735 | 0.798 | 0.952 | 0.873 | 0.937 | 0.891 | 0.843 | 1.049 |
| 0% HA | 0.993 | 0.9 | 0.853 | 0.768 | 0.792 | 0.796 | 0.853 | 0.803 | 0.829 | 0.904 | 0.804 | 0.999 |
| b-ground | 0.292 | 0.272 | 0.268 | 0.305 | 0.308 | 0.308 | 0.303 | 0.299 | 0.298 | 0.301 | 0.3 | 0.304 |

| | mean | S.D. | adj. | viability | S.D. |
|---|---|---|---|---|---|
| DMEM/FBS | 1.165333 | 0.128503 | 0.868833 | 66.9169796 | 9.897202 |
| 2% HA | 1.186167 | 0.142727 | 0.889667 | 68.5215494 | 10.99271 |
| 1% HA | 0.892917 | 0.14995 | 0.596417 | 45.9356247 | 11.54905 |
| 0.5% HA | 0.96225 | 0.208319 | 0.66575 | 51.275633 | 16.04458 |
| 0.1% HA | 0.9055 | 0.093065 | 0.609 | 46.9047848 | 7.167842 |
| 0% HA | 0.857833 | 0.07694 | 0.561333 | 43.2335291 | 5.925881 |
| b-ground | 0.2965 | 0.013174 | 0 | | |

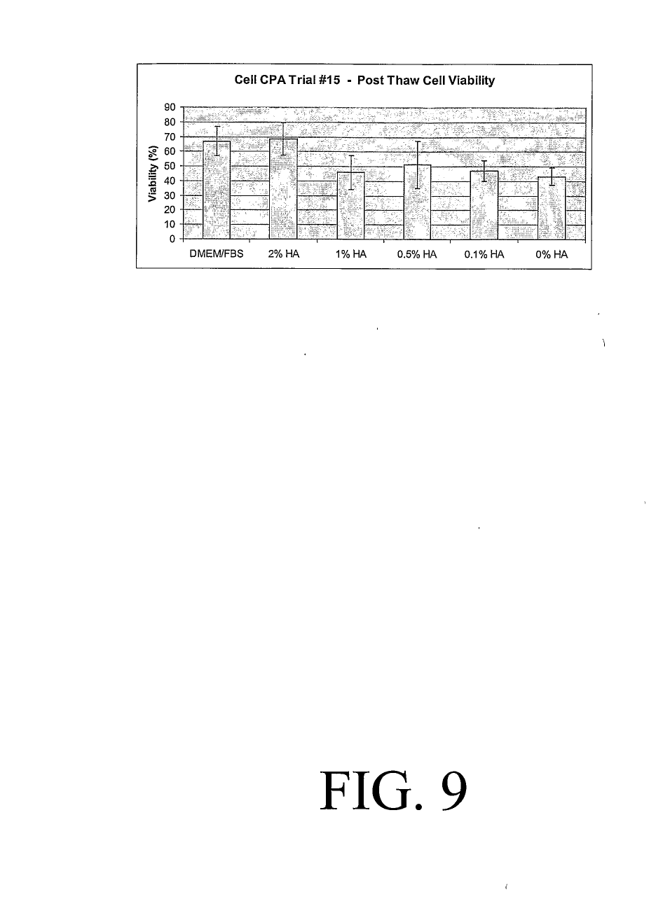

In another example, post-thaw WST data was collected at 60 minutes. Various concentrations of HA can be used in cryopreservation. For example, FIG. 9 shows that HA at concentrations of 0.1%, 0.5%, 1%, and 2% protect cell viability. Table 2 shows the results. The freezing an thawing protocols outlined above were used.

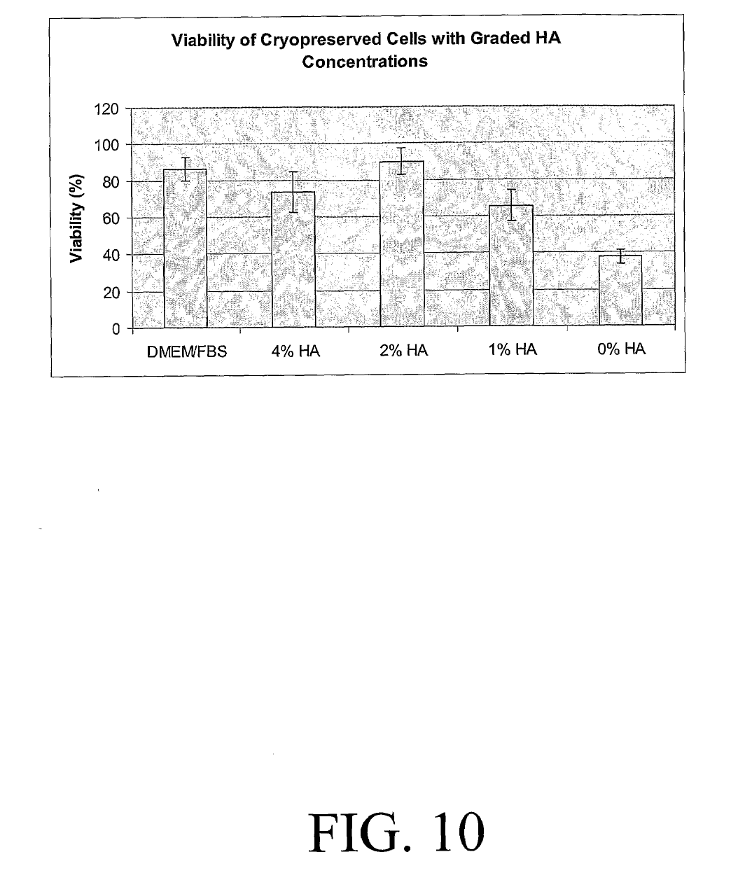

In another example, pre-freeze data was collected after 90 minutes of incubation, and post thaw data was collected after 90 minutes. The results (FIG. 10) show that various concentrations of HA can be used in fresh cell storage. HA can be used at concentrations of 1%, 2%, and 4% to protect cell viability. The freezing and thawing protocols described above were used. Table 3 shows the results.

TABLE 3
Cell CPA
Pre-freeze WST-1 data at 90 minutes incubation
MODE - Single
WAVE LENGTH - 450 nm
DATE - Nov. 02, 2004

| | | | | | | mean | adjusted |
|---|---|---|---|---|---|---|---|
| Fresh cells | 2.037 | 1.898 | 2.141 | 1.937 | 2.063 | 2.015 | 1.625 |
| Background | 0.389 | 0.385 | 0.389 | 0.385 | 0.403 | 0.39 | |

Post-Thaw WST-1 Data at 90 minutes
DATE - Nov. 04, 2004

| | | | | | | | | | |
|---|---|---|---|---|---|---|---|---|---|
| DMEM/ | 1.698 | 1.913 | 1.943 | 1.809 | 2.031 | 1.922 | 1.842 | 1.741 | 1.769 |
| FBS | | | | | | | | | |
| 4% HA | 1.385 | 1.61 | 1.621 | 1.539 | 1.888 | 1.567 | 1.885 | 1.806 | 1.465 |
| 2% HA | 1.788 | 2.041 | 2.012 | 1.906 | 1.904 | 2.034 | 1.985 | 1.865 | 1.68 |
| 1% HA | 1.373 | 1.586 | 1.605 | 1.613 | 1.585 | 1.558 | 1.462 | 1.623 | 1.234 |
| 0% HA | 0.97 | 1.068 | 1.089 | 1.052 | 1.13 | 1.111 | 0.975 | 1.129 | 1.056 |
| b- | 0.454 | 0.45 | 0.452 | 0.448 | 0.448 | 0.451 | 0.453 | 0.451 | 0.453 |
| ground | | | | | | | | | |

| | mean | S.D. | adj. | viability | S.D. |
|---|---|---|---|---|---|
| DMEM/FBS | 1.852 | 0.108 | 1.401 | 86.2085 | 6.664 |
| 4% HA | 1.641 | 0.181 | 1.19 | 73.2034 | 11.13 |
| 2% HA | 1.913 | 0.122 | 1.462 | 89.9487 | 7.485 |
| 1% HA | 1.515 | 0.134 | 1.064 | 65.4974 | 8.23 |
| 0% HA | 1.064 | 0.06 | 0.613 | 37.7436 | 3.666 |
| b-ground | 0.451 | 0.002 | 0 | | |

### 2. Example 2

In vitro Measurement of Viability of Stored and Cryopreserved Skin.

Data indicate that 1% or 2% w/v of 200 kDa HA can retain nearly complete physiological function of human epidermis at 7 and 10 days, a time point at which current cultured skin is no longer useable. Split thickness human skin consented for research is obtained within one hour of organ harvest, and immediately placed into media at 4° C. for transport. Within two hours the skin is cut into 8 mm diameter pieces, and placed into a 12 well plate containing RPMI-1640 plus HA. A reading for O₂ consumption is taken of the freshly harvested skin within 24 hours of harvest. The media is changed every two days, and at multiple days post-harvest, skin is assayed for O₂ consumption using an Orion Dissolved Oxygen probe/computer interface, with readings taken every 10 seconds continuously for 50 minutes.

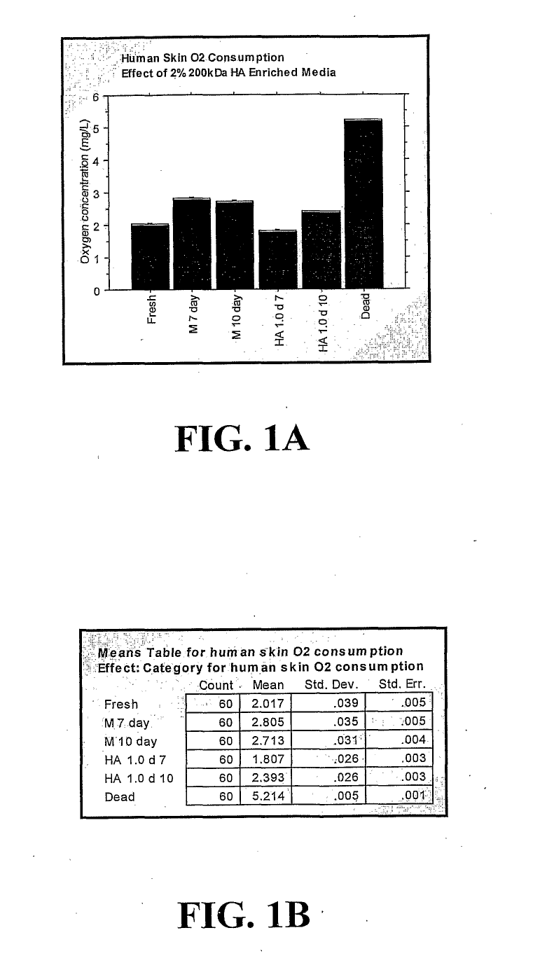

Data shown are the mean dissolved O₂ concentrations in the culture media, as shown in FIG. 1A and FIG. 1B, HA enriched media preserved viability of the human skin, assessed by O₂ consumption at 7 and 10 days post harvest (p<0.001 vs media controls). HA enriched media preparations, which show the greatest enhancement of skin viability, are used in culturing and storing skin at 4° C. prior to cryopreservation. Skin is taken from culture at 2, 5, 7 and 10 days and cryopreserved using standard tissue banking procedures, and kept at −80° C. Upon controlled thawing, the skin is placed into HA enriched media, and viability assessed by O₂ consumption assay at 24 and 48 hours. This information can guide composition and timing optimized for preparation of skin for cryopreservation.

### 3. Example 3

in vivo Assessment of Engraftment and Histologic Characterization.

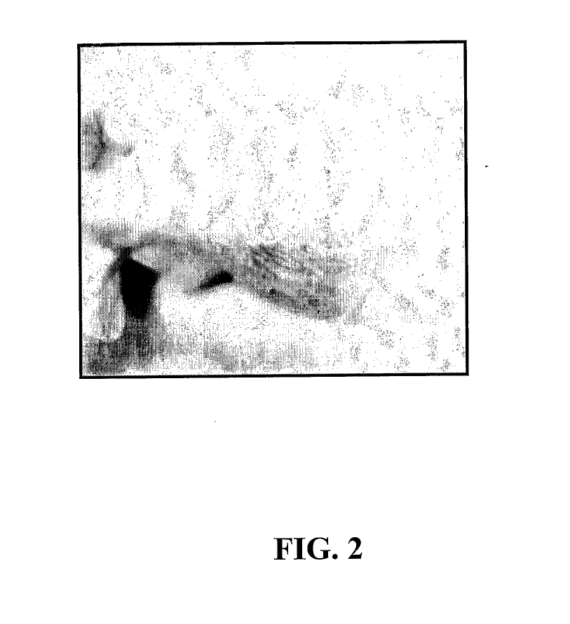

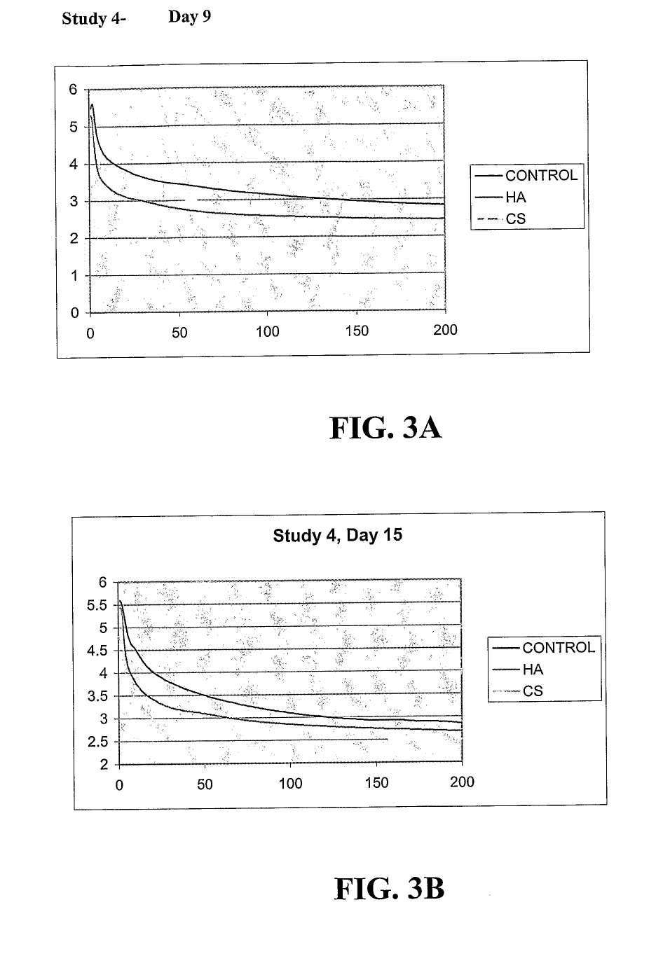

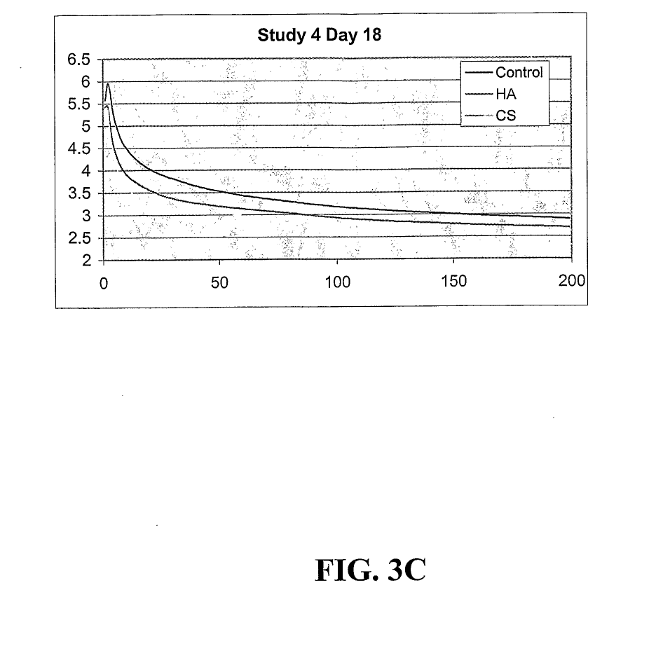

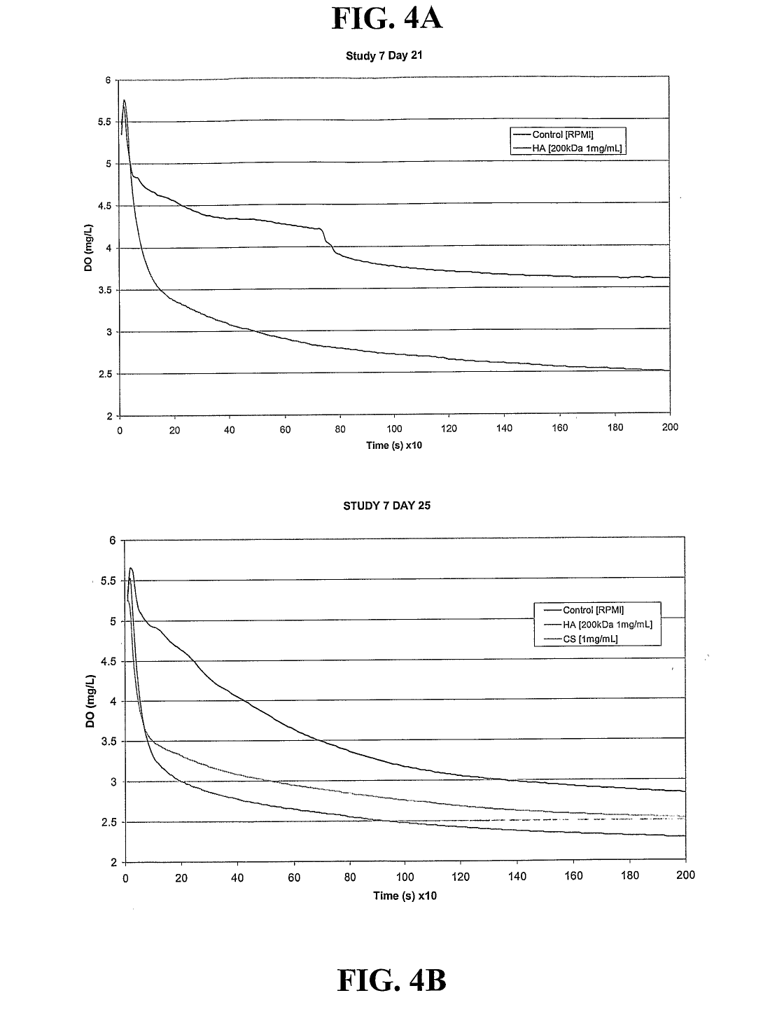

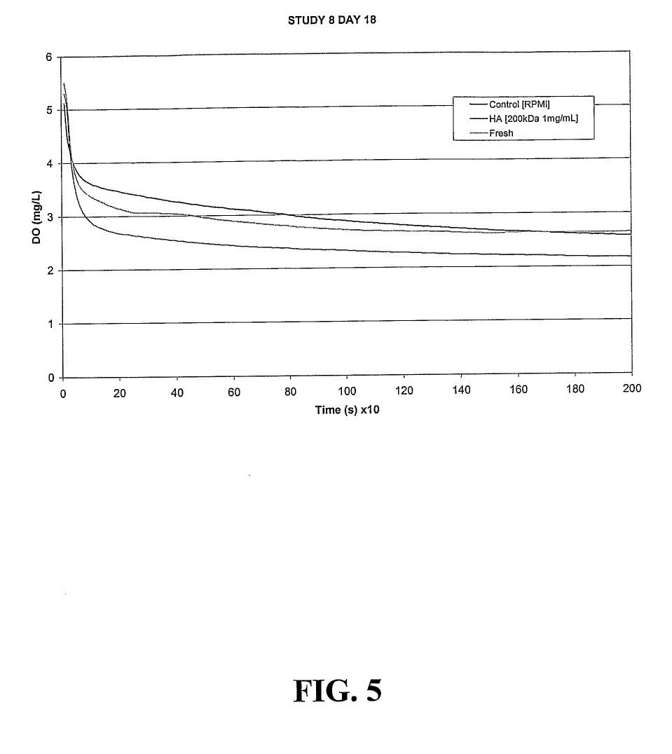

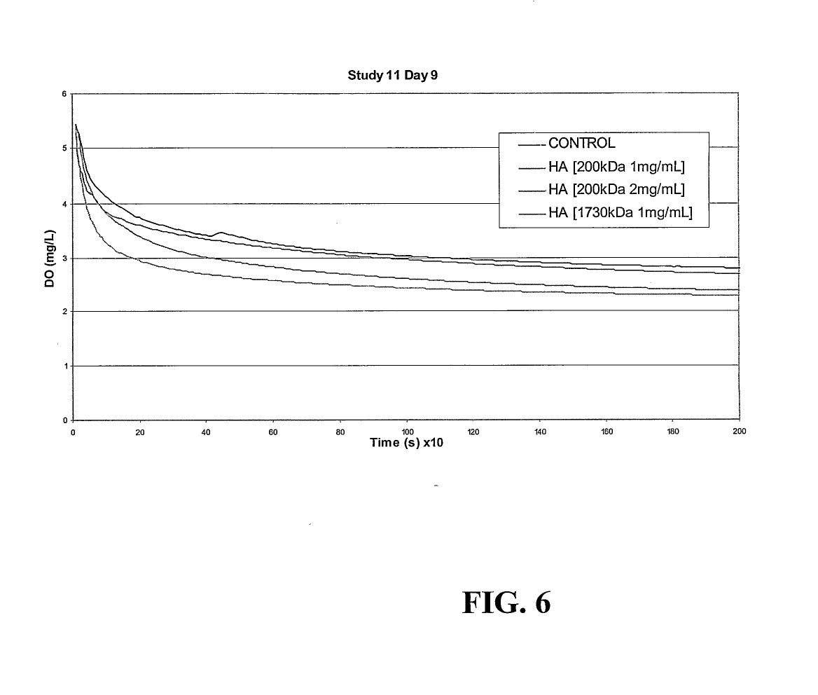

Sterile cadaveric human skin cultured in various media formulations for varied times post harvest (stored and cryopreserved), is cut to a size equal to the wound on the athymic nude mouse. This is a well-described model where human skin is not rejected and the graft maintains human skin characteristics (Cram A, Domayer M, Shelby J. Human skin storage techniques: a study utilizing a nude mouse recipient. 1983 J Trauma 23:924-6, Merrell S W, Shelby J, Saffle J et al. An in vivo test of viability for cryopreserved human skin. Curr Surg 43:296, 1986.) After the wound has been made, the cadaveric skin is immediately placed on the wound and sutured into place (FIG. 2). Laser Doppler ultrasound is used to determine cutaneous blood flow in healing skin, assessing potential faster engraftment of skin cultured in HA enriched media. Biopsies of the human skin graft are taken at 1, 2, and 4 weeks post transplant, and assessed histologically for structural integrity. Epithelial origin will be confirmed using direct immunofluorescence staining of healed graft epidermis with FITC-labeled monoclonal antibody against a common hapten of the HLA-ABC histocompatibility antigen (Boyce S T, Greenhalgh D G, Housinger T A, Kagan R J, Rieman M, Childress CP and Warden G D. Skin anatomy and antigen expression after burn wound closure, with composite grafts of cultured skin cells and biopolymers. 1993 Plast Reconstr Surg 91:632-41). This clinically relevant model provides definitive proof that the human donor epidermis survived storage and/or cryopreservation, and provides preclinical evaluation data of enriched media.

### 4. Example 4

### Assessment of HA as a Cryoprotectant Agent Compared to Other Cryoprotectants.

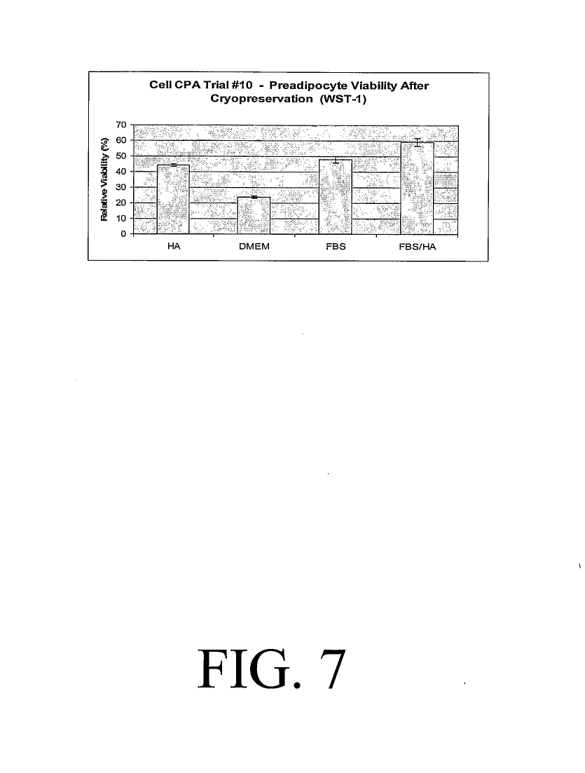

FIG. 7 shows HA is an effective cryoprotectant. A trial was conducted comparing the viability of preadipocyte cells after cryopreservation. The cells were stored in HA, DMEM, FBS, or FBS/HA. The cells were compared at pre-freeze, then again post-thaw. The results show that HA or FBS/HA was superior to DMEM alone. The results can be seen in Table 4 below. The protocol used for freezing/thawing follows.

### a) Freezing Protocol

Human preadipocytes were harvested from cadaveric donor. Cells were then cultured in FBS supplemented DMEM until the desired cell number was obtained. Cells were then harvested with 0.025% trypsin, with 1×10⁶ cells used for each experimental solution. The cells were first suspended with ½ mL of desired solution without DMSO, then the cell suspensions were cooled in an ice bath for 5 minutes. 250 uL of cryosolution containing 20% DMSO was added, and the cells were placed in an ice bath for 5 minutes. Additional 250 uL cryosolution with DMSO was added to obtain a total volume of 1 mL with final concentration of DMSO equal to 10%. The cryovials were keot on ice until cryopreserved with controlled rate freezer, then stored in a liquid nitrogen storage tank.

### b) Thawing Protocol

Cells were transferred to a −20° C. freezer for 30 minutes. Cryovials were then opened in a clean bench to release pressure. They were then rapidly thawed in 37° C. water bath only until the last ice was melted. They were then cooled in an ice bath for 5 minutes. ½ mL mannitol solution was added, and the cells were allowed to cool for 3 minutes. Additional ½ mL mannitol solution was added, and again allowed to cool for 3 minutes. The cells were then transferred to a 50 mL conical tube with 2 mL cold DMEM, and then placed in an ice bath for 2 minutes. 16 mL of cold DMEM was added, and the mixture returned to an ice bath for 2 minutes. The cells were then centrifuged for 5 minutes and the supernatant was aspirated. The cells were resuspended in 4 mL culture media, and the WST-1 viability assay was performed with 200 uL cell suspension and 20 uL WST-1 reagent.

TABLE 4
Cell CPA
Pre-Freeze WST-1 Data (90 min)

| | fresh | b- |
|---|---|---|
| | cells | ground |
| | 2.462 | 0.484 |
| | 2.249 | 0.489 |
| | 2.504 | 0.499 |
| mean | 2.405 | 0.490667 |
| St. dev. | 0.136722 | 0.007638 |
| adjusted | 1.914333 | 0 |

Post-Thaw WST-1 Data (90 min)

| | | | | | b- |
|---|---|---|---|---|---|
| | HA | DMEM | FBS | FBS/HA | ground |
| | 1.41 | 0.983 | 1.486 | 1.731 | 0.537 |
| | 1.382 | 1.015 | 1.412 | 1.645 | 0.548 |
| | 1.374 | 0.988 | 1.475 | 1.651 | 0.543 |
| mean | 1.388667 | 0.995333 | 1.457667 | 1.675667 | 0.542667 |
| St. dev. | 0.018903 | 0.017214 | 0.039929 | 0.048014 | 0.005508 |
| adjusted | 0.846 | 0.452667 | 0.915 | 1.133 | 0 |
| viability | 44.19293 | 23.64618 | 47.79732 | 59.18509 | |
| St. dev. | 0.987459 | 0.899234 | 2.085797 | 2.508126 | |

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

Throughout this application, various publications are referenced. The disclosures of these publications in their entireties are hereby incorporated by reference into this application in order to more fully describe the state of the art to which this invention pertains.

### F. REFERENCES

1. Fratianne R B, Brandt C P. Improved survival of adults with extensive burns. J Bum Care Rehabil 1997 Jul-Aug; 18(4):347-51
2. Gardner D K, Rodriegez-Martinez H and Lane M. Fetal development after transfer is increased by replacing protein with the glycosaminoglycan hyaluronan for mouse embryo culture and transfer. 1999 Hum Reprod 14 (10):2575-84
3. Stojkovic M, Thompson J G and Tervit, H R. Effects of hyaluronic acid supplementation on in bitro development of bovine embryos in a two-step culture system. 1999 Theriogenology 51: 254.
4. Tammi R, Saamanen A-M, Maibach H I and Tammi M. Degradation of newly synthesized high molecular mass hyaluronan in the epidermal and dermal compartments of human skin in organ culture. 1991 J Invest Dermatol 97: 126-130.
5. Poggi M M, Klein M B, Chapo G A, Cuono C B. Effects of cryopreservation and deconstruction on the dermal glycosamingoglycan content of human skin. 1999 J Burn Care Rehabil 20 (3):201-6.
6. Bravo D, Rigley T H, Gibran N, Strong D M, Newman-Gage H. Effect of storage and preservation methods on viability in transplantable human skin allografts. 2000 Burns 26(4):367-78.
7. www.biolifesolutions.com
8. www.ameriburn.com The American Burn Association
9. www.aatb.com The American Association of Tissue Banks
10. Fraser J R E, Laurent T C, and Laurent U B G. Hyaluronan: Its nature, distribution, functions and turnover. J. Intern. Med. 1997;242(1):27-33.
11. Collis L, Hall C, Lange L, Ziebell M R, Prestwich G D, and Turley E A. Rapid hyaluronan uptake is associated with enhanced motility: implications for an intracellular mode of action. FEBS Lett. 1998;440(3):444-449.
12. Hardwick C, Hoare K, Owens R, Hohn H P, Hook M, Moore D, Cripps V, Austen L, Nance D M, and Turley E A. Molecular cloning of a novel hyaluronan receptor that mediates tumor cell motility. J. Cell Biol. 1992;117:1343-1350.
13. Gerdin B and Hallgren R. Dynamic role of hyaluronan (HYA) in connective tissue activation and inflammation. J. Intern. Med. 1997;242(1):49-55.
14. Boyce D E, Thomas J H, Moore K, and Harding K. Hyaluronic acid induces tumour necrosis factor-□ production by human macrophages in vitro. British J. Plastic Surgery 1997;50:362-368.
15. Iocono J A, Kmmel T M, Keefer K A, Allison G M, and Paul H. Repeated additions of hyaluronan alters granulation tissue deposition in sponge implants in mice. Wound Repair Regen. 1998;6(5):442-448.
16. Moseley R, Leaver M, Walker M, Waddington R J, Parsons D, Chen W Y, Embery G. Comparison of the antioxidant properties of HYAFF-11p75, AQUACEL and hyaluronan towards reactive oxygen species in vitro. 2002 Biomaterials 23:2255-64.
17. Zieger M A J, Tredget E R, McGann L E. A simple, effective system for assessing viability in split-thickness skin with the use of oxygen consumption. 1993 J Burn Care Rehabil 14:310-18
18. Cram A, Domayer M, Shelby J. Human skin storage techniques: a study utilizing a nude mouse recipient. 1983 J Trauma 23:924-6.
19. Merrell S W, Shelby J, Saffle J et al. An in vivo test of viability for cryopreserved human skin. Curr Surg 43:296, 1986.
20. Boyce S T, Greenhalgh D G, Housinger T A, Kagan R J, Rieman M, Childress C P and Warden G D. Skin anatomy and antigen expression after burn wound closure, with composite grafts of cultured skin cells and biopolymers. 1993 Plast Reconstr Surg 91:632-41
21. Kirker K, Luo Y, Nielson J H, Shelby J, Prestwich G. Glycosaminoglycan hydrogel films for wound dressing. Biomaterials 23: 3661-3671, 2002

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed:

1. A composition comprising a cell, tissue, or organ and glucosaminoglycan in the absence of serum.

2. The composition of claim 1 wherein the glucosaminoglycan is hyaluronan.

3. The composition of claim 1 wherein the composition is for preserving cells.

4. The composition of claim 3 wherein the cells are non-cultured.

5. The composition of claim 3 wherein the cells are cultured and preserved at a temperature of above freezing.

6. The composition of claim 3 wherein the cells are not unkeratinized epithelial cells.

7. The composition of claim 3 wherein the cells are non-corneal.

8. The composition of claim 3 wherein the cells are non-ovarian.

9. The composition of claim 3 wherein the cells are non-musculoskeletal.

10. The composition of claim 3 wherein the cells are in the absence of a non-cell penetrating cryoprotectant.

11. The composition of claim 3 wherein the cells are preserved at a temperature above freezing.

12. The composition of claim 3 wherein the cells are preserved at a temperature below freezing.

13. The composition of claim 1 wherein the cells are epithelial cells.

14. The composition of claim 13 wherein the cells are non-cultured.

15. The composition of claim 13 wherein the cells are cultured and preserved at a temperature above freezing.

16. The composition of claim 13 wherein the cells are preserved in the absence of a non-cell penetrating cryoprotectant.

17. A method of making a cell-containing storage solution comprising
utilizing a solution comprising a glucosaminoglycan in the absence of serum; and
placing the cells in the solution.

18. The method of claim 17 wherein the glucosaminoglycan is hyaluronan.

19. A method of preserving cells comprising
utilizing a solution comprising glucosaminoglycan in the absence of serum; and
storing the cells in the solution.

20. The method of claim 19 wherein the glucosaminoglycan is hyaluronan.

21. The method of claim 19 wherein the cells are preserved at a temperature above freezing.

22. The method of claim 19 wherein the cells are preserved at a temperature below freezing.

23. A method of treatment comprising
obtaining cells;
storing the cells in a solution comprising glucosaminoglycan in the absence of serum; and
using the cells in treatment.

24. The method of claim 23 wherein the glucosaminoglycan is hyaluronan.

25. A kit comprising a storage solution comprising hyaluronan in the absence of serum and cells.

26. The kit of claim 25 wherein the cells are stored at a temperature above 0° C.

27. The kit of claim 25 wherein the cells are in sheets.

28. The kit of claim 25 wherein the cells are in solution.

29. The kit of claim 25 wherein the cells are a tissue.
